(19) United States
(12) Patent Application Publication
(10) Pub. No.: US 2021/0268215 A1
(43) Pub. Date: Sep. 2, 2021

(54) Smoking-Cessation Apparatus and Method

(71) Applicants: Joshua Israel, Belle Mead, NJ (US); Devin Serago, Sewell, NJ (US)

(72) Inventors: Joshua Israel, Belle Mead, NJ (US); Devin Serago, Sewell, NJ (US)

(21) Appl. No.: 16/806,871

(22) Filed: Mar. 2, 2020

(51) Int. Cl. A61M 15/00; A24F 40/30; A24F 40/42; A24F 40/51

(52) U.S. Cl. CPC A61M 15/0066; A61M 15/0068; A24F 40/51; A24F 40/42; A24F 40/30

(57) ABSTRACT

A smoking-cessation device and method includes a vaporization device and smoking-cessation method. The device includes two pods, each of which is a consumable, interchangeable component that contains a capsule tank with a measured amount of vaporizable liquid. Measures of a substance containing a drug such as nicotine may be delivered via instructions from a computer-readable medium to each pod as part of a smoking-cessation regimen. The regimen can be changed and reprogrammed by a medical practitioner to delivery varying amounts of drug according to a person's adherence to a defined smoking-cessation regimen. Eventually the drug dosage will be reduced, and the non-drug liquid will increase until the user reduces his or her dependence on the drug.

### TECHNICAL FIELD

The present disclosure relates generally to smoking-cessation methods and devices, and more specifically to vaporization devices used in smoking cessation.

### BACKGROUND

Vaporizer devices as cigarette alternatives are known in the art. These devices are usually battery-operated, handheld devices configured to simulate smoking a cigarette. Rather than burning tobacco, some of these devices heat liquid solutions that can be inhaled and exhaled in a vapor. Such liquid solutions may deliver varying amounts of nicotine or other drugs.

Vaporizer devices usually comprise a device body and a cartridge. The body usually employs an electronic heating assembly adapted to heat an evaporable material and to produce an inhalable vapor.

In some devices, two heater contacts each have a plate coupled to each side. Device heaters usually have a wick and a resistive heating element in contact with the wick and the two plates. The device body usually comprises a cartridge receptacle that, once inserted into the device body, couples its heater contacts with those in the body. An LED indicator shows device's state.

An externally insulated, battery-powered heater surrounds the vaporization chamber of an exemplary device. The vaporization chamber is typically covered by a removable mouthpiece. A microcontroller usually regulates temperature in the vaporization chamber.

Bluetooth Low Energy (Bluetooth LE or BLE) is a wireless personal area network technology that uses less power than Classic Bluetooth while operating in a similar range. It is used in healthcare, fitness, security, and home entertainment devices.

Ambient backscatter technology uses existing TV and cellular signals to provide the power and medium for battery-less communications. Devices using an ambient backscatter system have antennas that pick up TV or cellular signals and convert them into electricity, which are then reflected to and received by other devices with similar antennas. Ambient backscatter technology lets devices communicate without being turned on.

Near-field communication (NFC) is a set of communication protocols that enable two electronic devices to establish communication by bringing them within 1.5 inches of each other. NFC devices are used in contactless payment systems, similar to those used in credit cards and electronic ticket smart cards and allow mobile payment to replace or supplement these systems.

ZigBee is a specification for a suite of communication protocols used to assemble personal area networks with small, low-power digital radios. It is used in home automation, medical-device data collection, and other low-power, low-bandwidth needs, designed for small scale projects which need wireless connection. ZigBee is a low-power, low-data-rate, and close proximity (i.e., personal area) wireless, ad-hoc network. Applications include wireless light switches, home energy monitors, traffic-management systems, and other consumer and industrial equipment that requires short-range, low-rate wireless data transfer. It can transmit over distances 10-100 meters in line-of-site, and over longer distances through a mesh network.

Polyphenylene sulfide (PPS) is a semi-crystalline polymer that can withstand flame heat and resist chemical treatment. It is used in automotive, electronic and mechanical parts.

Polyether ether ketone (PEEK) is an organic thermoplastic polymer used in engineering applications in automotive, aerospace and other industries.

In the context of this disclosure “substance” refers to a drug or supplement. For clarity, the apparatus and method of the disclosure may refer to the use of nicotine as a substance in a vaporizable liquid to be vaporized and inhaled. Nicotine is used as a substance in the instant example embodiment, and not intended to be limiting. In this disclosure “inert ingredients,” “inert substance” or “inert vapor” refers to a vaporizable substance that does not contain a drug. It may contain flavorings and the like.

### SUMMARY

A smoking-cessation method and device includes a vaporization device and smoking-cessation method.

The device has a first body, also referred to as a pod assembly, and a second body, also referred to as a base assembly.

The first body has a top, bottom, interior and exterior. The top exterior is a mouthpiece or cover that has in its center an orifice for inhalation. In the interior, a pod assembly comprises a first pod, a second pod, and a mixing chamber. Each pod has at least one wick. Each pod is a consumable, interchangeable component. The pods' upper sections reside in the first body and their lower sections reside in the second body (the base assembly). The pods are in fluid and electrical communication with the base assembly. Each pod holds a capsule tank which contains a measured amount of vaporizable liquid. Vaporizable liquid in each cartridge is moved by capillary action into each wick. Each wick is connected to a heating coil that when heated produces vapor. Each wick is disposed proximal to the orifice. Vapor is pulled through the orifice during inhalation.

The second body has a top, bottom, interior and exterior. The top of the exterior is a housing into which the first body is inserted, so that the bases of the pods are held partially in the second body. The interior of the second body holds electronic components. including, a battery, a printed circuit board, a flexible printed circuit, and a pressure sensor.

A firewall gasket is disposed between the pod assembly and the base assembly. The firewall gasket provides a sealed transition for electrical connections and connects the atmospheric pressure sensor to the pod assembly via a conduit. An air-intake orifice provides a relatively narrow inlet for incoming air that passes the conduit. The atmospheric pressure sensor senses a change in pressure caused by the user inhaling through the air-intake orifice, and signals the microprocessor to initiate the vaporization process.

A processor on the printed circuit board controls each heating coil individually. A defined voltage over a coil for a set time produces vapor. Specific amounts of a substance such as nicotine may be measured and dispensed in vapor form as part of a smoking-cessation regimen.

The atmospheric pressure sensor measures changes in pressure in order to count the number of times the apparatus is used. A secondary conduit extends from the pressure sensor on the printed circuit board, into the pod assembly where it measures atmospheric pressure changes that occur in the pod assembly when a user reduces pressure via inhalation through the mouthpiece. Changes in atmospheric pressure are measured and recorded as a means of counting the number of inhalations or “hits.” A substantial change in atmospheric pressure may be understood to connote inhalation of vapor from the apparatus.

Some embodiments employ a method for facilitating smoking cessation. In these embodiments a processor stores machine-readable instructions that control the amount of vapor produced from each pod in each series of hits. Initial hits may include mostly vapor from the nicotine-containing cartridge and comparatively less vapor from the cartridge containing a vaporizable liquid without nicotine; eventually the amount of nicotine vaporized will be reduced and the amount of non-drug liquid vaporized will increase until the user reduces his or her dependence on nicotine. This gradual reduction of nicotine is referred to as regression. In one embodiment, regression is controlled according to a prescribed regimen of drug/non-drug liquids that is configured and administered by a physician or other administrator. One skilled in the art understands that any vaporizable drug-containing liquid may be used in this apparatus and method; nicotine is used here as an example.

In some embodiments the duration of a hit (or “hit duration,”) is measured in seconds; this duration is preferably two seconds, but may be between 0.5 and 4 seconds. In an example embodiment, a cessation regimen may enable two-second hits during the regimen's control period. In this period the heating element in the nicotine-containing pod is activated for two seconds, while the heating element in the inert-ingredients pod is not activated.

A regression plan is input to the device's microcontroller as machine-readable instructions. The plan may, during the two seconds of a hit, reduce the amount of heat to a heating element in the nicotine-containing pod while increasing the amount of heat to the heating element in the inert-ingredients pod.

An example regimen includes an initial period during which no regression takes place. Throughout the regimen, following the initial period, the nicotine dosage changes daily according to a mathematical formula that drives computer-readable instructions that control the apparatus to deliver less nicotine and more inert ingredients over time. The duration of each hit remains constant throughout the regimen.

In another embodiment the above-mentioned prescription may accommodate a limited-duration deviation from the regimen. For example, a user may customarily take a given number of hits per day and may be in the 20th day of a 90-day regimen. If for example on the 20th day a user takes considerably more than the customary number of daily hits, the program may accept the deviation and initiate adjustment by alerting a physician or other administrator. The administrator may, in this example, extend the end date to accommodate the deviation, and/or alter the dosage.

In another embodiment, a program alters a regimen to account for a deviation. It does so by looking up the number of days on which the number of hits deviated substantially from the average, and adding that number of days to the duration of the regimen.

### BRIEF DESCRIPTION OF THE DRAWINGS

FIG. 1 is a top perspective view depicting an example embodiment;

FIG. 2 is a perspective, partially exploded view of the embodiment of FIG. 1;

FIG. 3a is an exploded view of a first portion of the embodiment of FIG. 1;

FIG. 3b is a detailed bottom view of the first portion of the embodiment of FIG. 1;

FIG. 4 is an exploded view of a second portion of the embodiment of FIG. 1;

FIG. 5 is a diagram illustrating operations associated with the use of the apparatus of FIG. 1.

FIG. 6 is a diagram of a method of using the apparatus of FIG. 1.

FIG. 7 is a graph of the linear equation of the method of FIG. 6.

FIG. 8 is a diagram of a method of using the apparatus of FIG. 1.

FIG. 9 is a graph of the exponential equation of the method of FIG. 8.

### DESCRIPTION

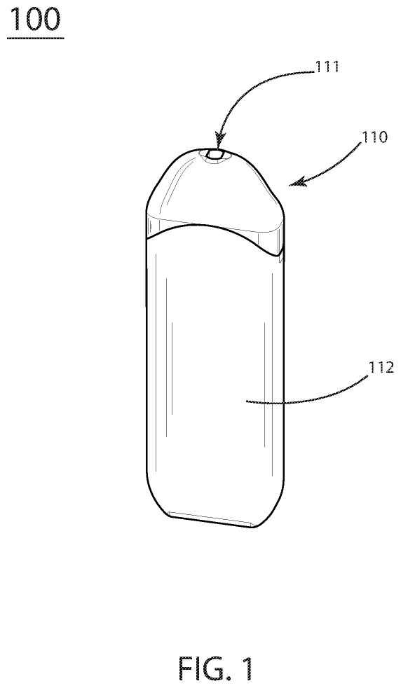

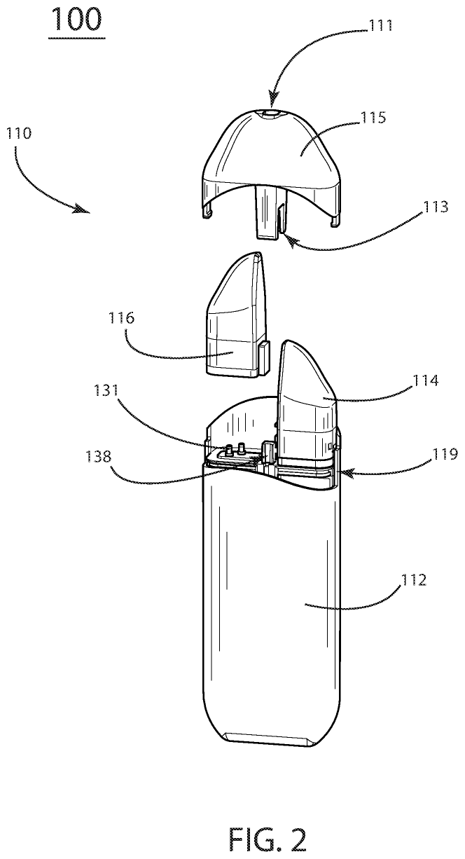

In FIGS. 1 and 2, a housing 112 holds at least electronic components and a battery. A pod assembly 110 has pods 114 and 116 that are partially housed in a mouthpiece 115 having an orifice 111 for inhaling through. In an example embodiment a mouthpiece 115 houses a first pod 114 and a second pod 116.

Referring to FIG. 2, in some embodiments, a first pod 114 is adjacent to a second pod 116 in the mouthpiece 115 and is located at least in part in the housing 112. Pods 114 and 116 are consumable components that may be inserted and removed by a user. Each pod 114/116 operates independently and may be replaced independently. Each pod 114/116 is in fluid communication with a primary conduit 113 where vapor transitions from a heating coil and wick combination that produces vapor that is inhaled through the orifice 111. An opening 119 in the housing 112 provides an inlet for air that passes a secondary conduit 138 before passing through the primary conduit 113 and being inhaled by the user through the orifice 111. The secondary conduit 138 is in fluid communication with a pressure sensor that initiates power to the independent coil-and-wick combinations to produce vapor according to an associated regimen.

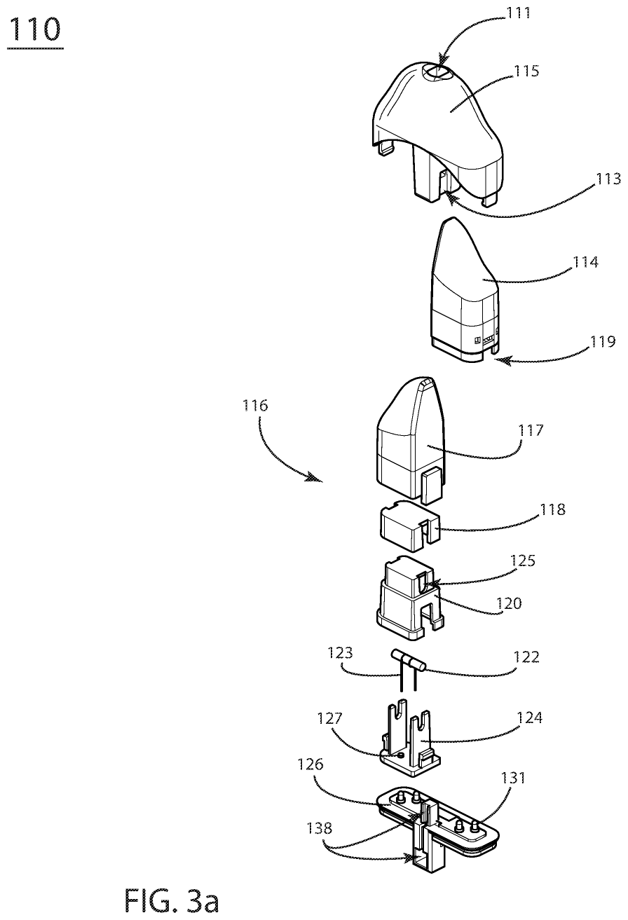

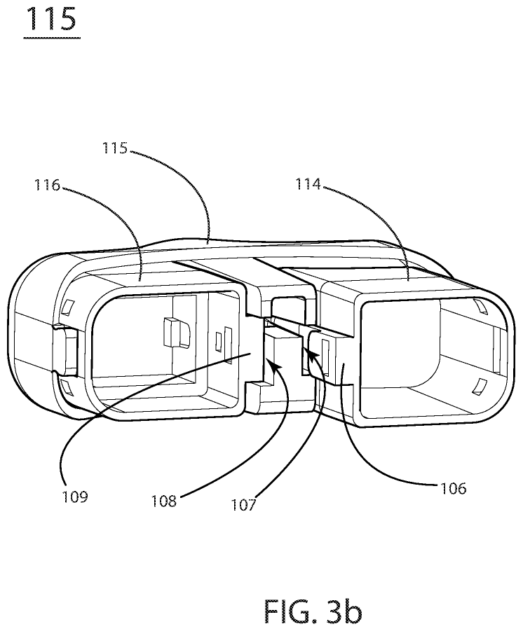

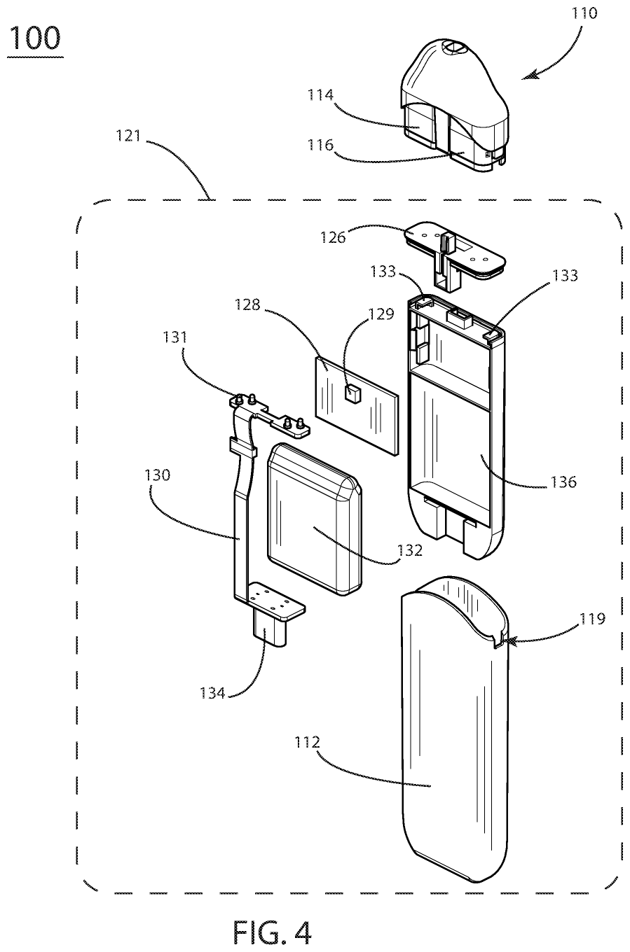

FIG. 3a is an exploded view of the pod assembly 110 showing a firewall gasket 126 for context. The mouthpiece 115 has an orifice 111 for inhaling vapor therethrough. The orifice 111 is in fluid communication with a primary conduit 113 that is in fluid communication with the air intake 119. In the illustration, pod 114 is shown assembled and pod 116 is shown in exploded view. A capsule tank 117 contains a vaporizable liquid. A capsule gasket 118 seals the vaporizable liquid in the capsule tank 117. One skilled in the art understands that a gasket may be made of rubber, silicone rubber or castable elastomeric polymers. A capsule insert 120 provides a structure to hold the capsule gasket 118 in place and also provides an orifice 125 for receiving a wick 122. The wick 122 is supported by a capsule base 124 that holds the wick 122 in place proximal to the orifice 125 in the capsule insert 120. The capsule base 124 further supports a heating element, also referred to as a coil 123 and associated electrical contacts 127 that make electrical contact with electrical connection structures 131 in the firewall gasket 126, which in turn are in electrical communication with a printed circuit board 128 (FIG. 4) and powered by the battery 132 (FIG. 4). The capsule base is affixed to the bottom of the capsule tank 117 and secures the capsule insert 120 in place while also pressing the capsule gasket 118 so as to make a liquid-tight seal. A secondary conduit 138 is in fluid communication with a pressure sensor 129 (FIG. 4) on the printed circuit board 128 (FIG. 4), and extends to the top surface of the firewall gasket 126 continuing into the interior of the pod assembly 110 (FIG. 4) where it measures changes in atmospheric pressure therein.

Vaporizable liquid is drawn by capillary action through the wick 122 (FIG. 3a), which is in fluid communication with the interior of the capsule tank 117 and in physical contact with the coil 123 that when heated produces vapor. One skilled in the art understands that a pressure sensor is used to detect a user's inhalation through the device. The change in measured pressure initiates a sequence in which electrical energy is directed through a coil.

One skilled in the art understands that a specific voltage over a coil 123 for a specific time will produce an amount of heat to produce a replicable amount of vapor. In this manner measured doses of a substance such as nicotine may be delivered in a vapor.

FIG. 3b illustrates a detail which makes up a portion of the first portion 115 of the embodiment 100. Pod 116 includes a protrusion 109 that fits in a slot 108 in the mouthpiece 115. Another pod 114 includes a protrusion 106 that fits into a slot 107 in the mouthpiece 115. The protrusion 109 on pod 116 that contains nicotine is larger than the protrusion 106 on the pod 114 that contains inert ingredients. In this example a pod containing nicotine having a protrusion as large as the example protrusion 109, would not fit in the slot 107. In this manner a pod containing inert ingredients could not be replaced with a pod containing nicotine.

FIG. 4 is a partially exploded view that illustrates the primary components of a base assembly 121. A pod assembly 110 is in fluid and electrical communication with the base assembly 121. The pod assembly is illustrated in detail in FIG. 3a.

In the base assembly 121 (FIG. 4), a firewall gasket 126 provides a sealed barrier between the interior of the housing 112 and the pod assembly 110 and seals around electrical-connection structures 131. The firewall gasket 126 includes a conduit between the pressure sensor 129 and the pod assembly 110. One skilled in the art understands that a pressure sensor may be used to measure changes in atmospheric pressure. Inhalation by user results in a change in atmospheric pressure inside the pod assembly 110. Inhalation or drawing of vapor from the pod assembly may be referred to as a use or a hit. A means of counting hits is by measuring and recording changes in atmospheric pressure. Upon sensing a change in pressure the atmospheric pressure sensor signals the control circuitry to initiate the heating of at least one coil to produce vapor according to a smoking-cessation regimen.

A housing insert 136 is a structure for containing a printed circuit board 128, a battery 132 and a flexible printed circuit 130. The housing further provides an attachment 133 for the pods 114/116. The pods 114/116 may attach by a snap fit, magnetic contact or similar means of removable attachment 133. The flexible printed circuit 130 joins the charge port 134 to the printed circuit board 128 for charging the battery 132, and connects the printed circuit board 128 to the electrical connection structures 131 that power the coils which heat the wicks 122 (FIG. 3a). A processor on the printed circuit board 128 regulates power to each heating coil individually; in this way the system controls the amount of vapor produced from each pod independently.

Some embodiments offer a method of facilitating smoking cessation by gradual reduction in nicotine delivery. A processor stores machine-readable instructions that control the amount of vapor produced by each pod in each series of hits. Initial hits may include mostly vapor from a first pod 114 which in this example stores a vaporizable liquid containing nicotine; and comparatively less vapor from a second pod 116, which in this example contains a vaporizable non-nicotine liquid. Over a period of time the amount of nicotine-containing vapor of each hit supplied by the liquid in the first pod 114 decreases, while the amount of nicotine-free vapor, supplied by the liquid in the second pod 116, increases. In one example a hit lasting two seconds delivers 1.8 seconds of vapor from the nicotine-containing pod and 0.2 seconds of non-nicotine-containing vapor from the other pod. Over time the sequence progresses until a hit lasting two seconds has no nicotine-containing vapor and 100 percent non-nicotine-containing vapor.

One skilled in the art understands that the physical habit of taking a number of hits per day may remain substantially unchanged while the amount of nicotine contained in each hit may be gradually reduced over time. The gradual reduction of the substance—in this example, nicotine—is referred to as regression. In one embodiment, regression occurs according to a regimen that is prescribed and administered by a physician.

In yet another embodiment the above-mentioned prescription may accommodate a limited-duration deviation from the regimen. For example, a user may customarily take a given number of hits per day and may be in the n^{th} day of a 90-day regression. If for example on the 20th day a user takes considerably more than the customary number of daily hits, the program may accept the deviation and adjust to accommodate the deviation by alerting a physician or other administrator. The administrator may, in this example, restart the regimen according to the instructions for the most recent day on which the average number of hits (or substantial equivalent) were taken, and extend the end date to accommodate the deviation. In one embodiment, the program alters a regimen to account for a deviation. It does so by counting the number of days on which the number of hits deviated substantially from the average, and adding that number of days to the duration of the regimen.

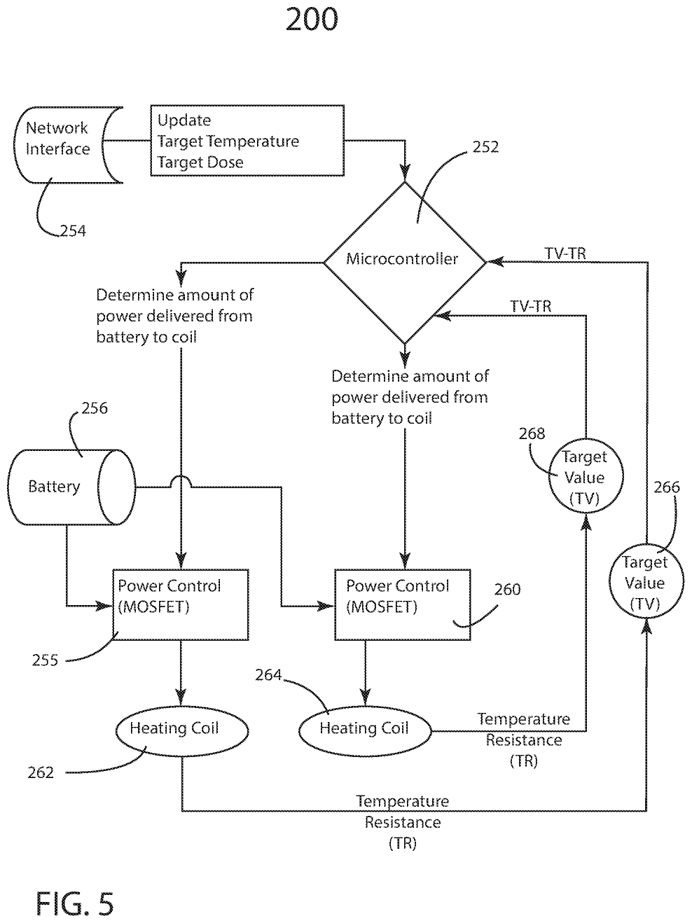

FIG. 5 is a block diagram 200 illustrating operations of example embodiments. The diagram shows a microcontroller 252, a network interface 254, a battery 256, a first power control 255, a second power control 260, a first heating coil 262, a second heating coil 264, a first target temperature value 266, and a second target temperature value 268.

A microcontroller 252 stores machine-readable instructions that determine an amount and duration of power to be delivered from the battery 256 to each heating coil 262/264 to reach target values. Via network interface 254, a user may update these target values by inputting new values, which are read by a microcontroller 252.

A first power controller 255 controls an amount of power to a first heating coil 262 according to instructions received from the microcontroller 252. Temperature resistance is measured in the coil, and the target value 266/268 is recognized by the microcontroller, which calculates the difference (TV-TR). The process loops, thus measuring the power delivered to the coil as well as the heat generated by the coil. A second power controller 260 controls the amount of power provided to a second heating coil 264 according to instructions received from the microcontroller 252. Power and heat are similarly controlled in the second heating coil 264.

An example regimen employs a linear formula which includes a baseline period (during which no regression occurs), followed by a period of regression which provides a series of changing daily doses. A regression formula is as follows:

tₑ=d₀/m+(t₀+c)

Where (d₀) is the initial dose and (t₀) is the start date of the cessation regimen, and (c) is the control period. Where (m) represents a constant that determines the slope of a regression curve. The higher the absolute value of (m), the more rapid the regression. If the end date (tₑ) is changed, (m) is changed. Alternatively, if a control period (c) is changed from a 90-day period to a 30-day period, the slope (m) will become steeper.

The nicotine dosage changes daily throughout the regimen. The nicotine dose for the n^{th} day is calculated according to the following formula:

dₙ=d₀−m*(tₙ−(t₀+c))

Where (dₙ) is the current dose and (tₙ) is the n^{th} day of the program.

The program changes the dosage in only the nicotine-containing pod. For this reason the pods are independently replaceable.

In an example program, during the first phase of a smoking-cessation regimen, the nicotine dosage in the nicotine pod will preferably diminish over time as a user is weaned from nicotine dependence. Accordingly, that cartridge will empty sooner than the non-nicotine cartridge. Toward the final phase of the regimen, the non-nicotine cartridge will empty more quickly than the nicotine-containing cartridge.

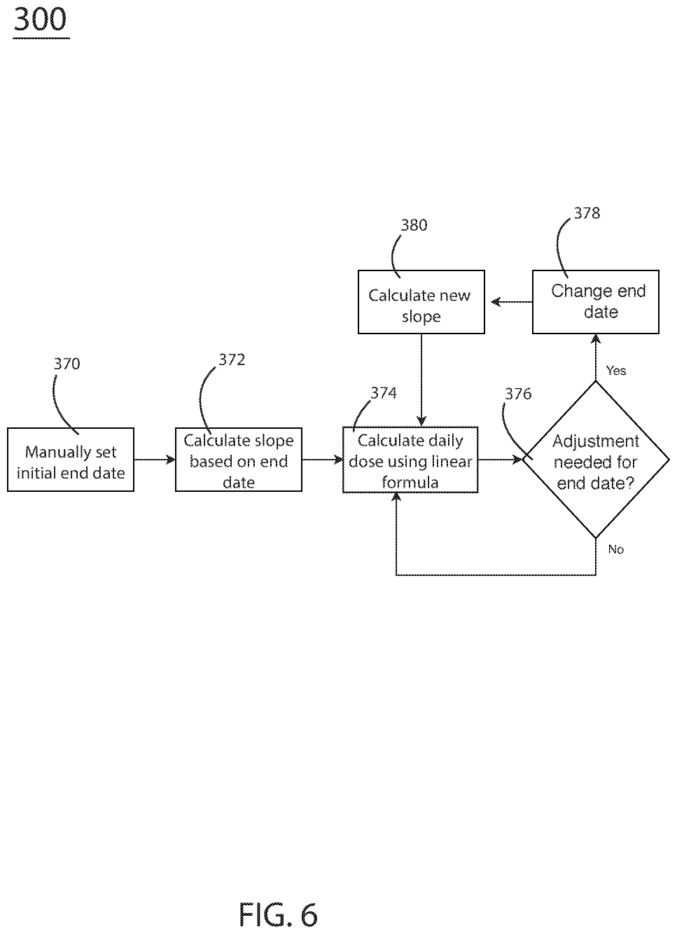

FIG. 6 illustrates an example embodiment of a set of machine-readable instructions for a method of gradually reducing a dose delivered in a vapor. The end date is manually set in a graphical user interface 370. The program calculates the slope of the line that denotes a regression 372 which is based on an end date. The program calculates a daily dose using the aforementioned linear formula 374. The program queries whether it is necessary to adjust 376 the end date of the regimen. If so, the program calculates a new slope 380 based on a new end date 378. If the program determines that no adjustment is needed, it continues calculating the daily dose according to the linear formula 374.

In another embodiment, the program calculates a daily dose using the aforementioned formula 374 based on number of hits. If the number of hits in the previous day is substantially equivalent to the average number of hits measured during the control period, the program continues. If the number of hits in the previous day is substantially greater than the average number of hits measured during the control period, the program changes the end date 378 by the number of days of deviation, and adds that number of days to the end date. The program then calculates a new slope 380 based on the new end date according to the linear formula.

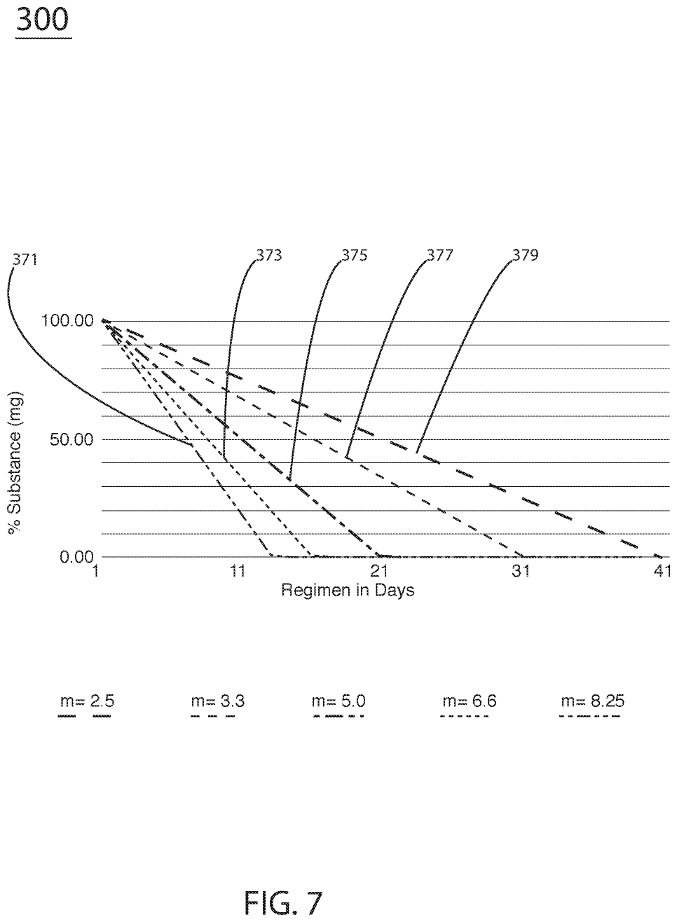

FIG. 7 is a graph illustrating various solutions to the aforementioned linear formula. Line 371 has a value for m=8.25 that generates a cessation plan having a duration of 13 days. Line 373 has a value for m=6.6 that generates a cessation plan having a duration of 16 days. Line 375 has a value for m=5.0 that generates a cessation plan having a duration of 21 days. Line 377 has a value for m=3.3 that generates a cessation plan having a duration of 31 days. Line 379 has a value for m=2.5 that generates a cessation plan having a duration of 41 days.

An exponential regression algorithm provides a regimen having a non-linear regression schedule. An exponential regression algorithm uses the following formula:

Dₙ=D₀*e^{k}(tₙ−t₀−c)

Where Dₙ is the nicotine dose on the n^{th} day; D₀ is the initial nicotine dose; e is a mathematical constant that is the base of the natural logarithm and is raised to the power of k, and where k is a constant that determines the slope of the graph that depicts the regression of the regimen. (tₙ) is the n^{th} date of the program. The start date, i.e., when a user begins the nicotine-cessation regimen, is denoted by (t₀).

A control period (c) represents the length of the initial period during which a baseline is established. During the control period a particular dose (D₀) is delivered with each hit. In this manner the amount of nicotine in each hit is reduced according to a non-linear equation. In one example, k is a negative value. The greater the absolute value of k, the steeper the curve that governs the regression. In other words, a regression program with a k value of −0.15 will have a more gradual regression than that of a regression program with a k-value of −0.25. A regression program having a k-value of −0.25 is said to have a relatively steeper regression than that of regression program having a k-value of −0.15. In some embodiments the amount of nicotine use during the control period is used to set the value of k.

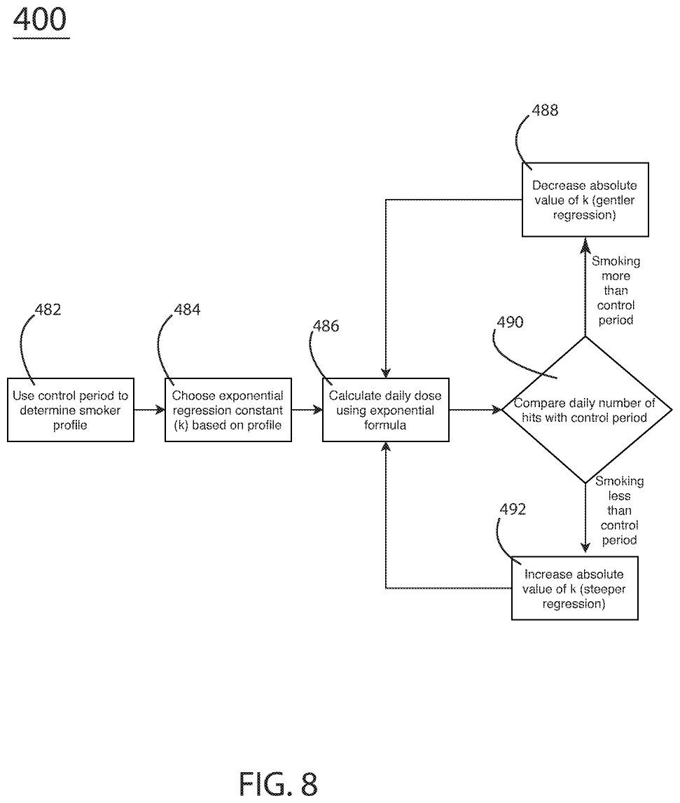

FIG. 8 illustrates an example embodiment of a set of machine-readable instructions, using the aforementioned formula, for a method of gradually reducing a dose by way of an exponential regression algorithm. The control period is used to determine the smoker profile 482 which in turn is used to determine the pace of the regression regimen. An exponential regression constant (k) is chosen based on the profile 484. The program calculates the daily dose using the exponential formula 486. The daily number of hits is compared with the number of hits taken by the user during the control period 490. Taking more hits is described in the illustration as “smoking more.” If the user takes a greater number of hits than during the control period (referred to in the illustration as “Smoking more than control period”), the program will decrease the absolute value of (k) to provide a more gradual (gentle) regression 488. The program will then calculate the daily dose using the (adjusted) exponential formula 486. If the user takes fewer hits (described in the illustration as “smoking less”) than during the control period, the program will increase the absolute value of (k), thus making a steeper, more rapid regression 492.

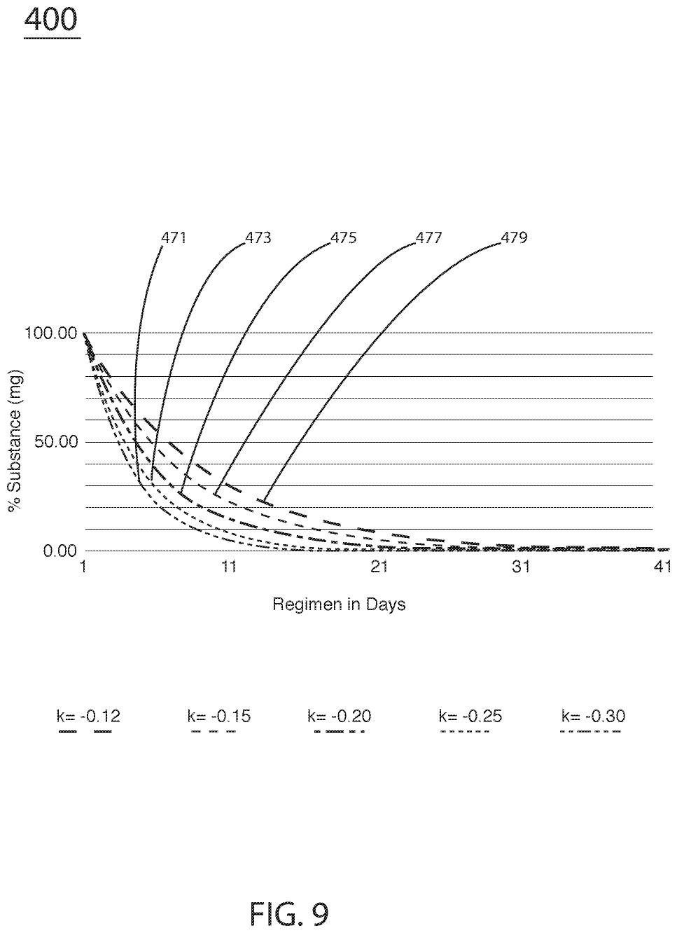

FIG. 9 is a graph illustrating various solutions to the aforementioned exponential formula. Line 471 has a value fork=−0.30 that generates a cessation plan that tapers to zero after 15 days. Line 473 has a value for k=−0.25 that generates a cessation plan that tapers to zero after 21 days. Line 475 has a value for k=−0.20 that generates a cessation plan that tapers to zero after 25 days. Line 477 has a value for k=0.15 that generates a cessation plan that tapers to zero after 31 days. Line 479 has a value for k=−0.12 that generates a cessation plan that tapers to zero after 41 days.

These descriptions demonstrate example embodiments and are not intended to be limiting.

1. A method and apparatus for ceasing use of a substance comprising:
a first body defining a first interior volume; and
a first pod and a second pod disposed within said first interior volume; and
a first conduit fluidly engaged with said first pod and said second pod and further fluidly engaged with the exterior of said first body through an orifice for inhaling therethrough; and
said first pod containing an amount of a vaporizable liquid containing a substance; and
said second pod containing an amount of vaporizable liquid containing inert ingredients; and
said first pod having a first wick coupled with a first heating element for creating a vapor from said liquid containing vaporizable substance;
and said second pod having a second wick coupled with a second heating element for creating a vapor from said liquid containing inert ingredients; and
said vapor from vaporizable liquid containing a substance and said vapor from said liquid containing inert ingredients movable through said first conduit; and
a second body defining a second interior volume; and
a power source disposed within the second body; and
a microcontroller in selective communication with a client computing device, the microcontroller storing machine-readable instructions from the client computing device for controlling power control circuitry; wherein
said power control circuitry is controlled by said microcontroller to provide varied power to said first heating element and said second heating element for the purpose of controlling the amount of vaporizable substance vaporized and the amount of inert ingredients vaporized for controlling the dosage of said substance for inhaling; and
said machine-readable instructions instructing a gradual reduction in the dose of said vaporizable substance while instructing a gradual increase in the amount of vaporizable, inert ingredients over time.

2. The apparatus and method of claim 1 further comprising:
said machine-readable instructions including:
instructions to vaporize said liquid containing vaporizable substance, for a period of less than a set hit duration; and
instructions to vaporize said liquid containing inert ingredients for the remainder of set hit duration.

3. The apparatus of claim 1 further comprising:
a target value of voltage and current for said first heating element; and
a target value of voltage and current for said second heating element; and
a resistor for measuring temperature resistance in said first heating element; and
a resistor for measuring temperature resistance in said second heating element, each electronically engaged with said microprocessor for providing a feedback loop for determining the temperature and duration of heat applied to said first wick and said second wick; wherein
the amount of heat from each heating element is sufficiently controlled so as to provide a specific volume of vapor and a known quantity of substance in said specific volume of vapor from said vaporizable liquid containing a substance.

4. The apparatus of claim 1 further comprising:
an atmospheric pressure sensor engaged with said microcontroller; and
a second conduit in fluid communication between said atmospheric pressure sensor and said first body for measuring changes in atmospheric pressure in said first body;
wherein
a substantial change in atmospheric pressure denotes inhalation through the apparatus, and each event of inhalation through the apparatus is counted as one hit.

5. The apparatus of claim 4 further comprising:
a non-transitory, computer-readable medium storing instructions in said microcontroller, recording changes in atmospheric pressure as measured by said atmospheric pressure sensor; and
a substantial change in atmospheric pressure in said first body denotes the inhalation of vapor from said apparatus; and each event of inhalation of vapor is counted as one hit; and
the average number of hits per day during the control period is measured; and
a significant increase in the number of hits in one day above said average number of hits triggers an event; wherein
non-transitory computer-readable medium storing instructions in said microcontroller recording said event and sending a notification to an administrator.

6. A method for ceasing use of a substance using the apparatus of claim 3 comprising:
determining an initial dose of a substance; and
determining a control-period duration; and
delivering, for the duration of said control period, said initial dose by using the apparatus of claim 1 for the purpose of:
wicking an amount of said liquid containing a substance; and
heating said amount of liquid containing a substance in said wick until it is vaporized; and
continuing said heating until the amount of substance vaporized is equal to said initial dose; and
setting a number of days for cessation; and
dividing said initial dose by said number of days for cessation to determine a regression constant; and
beginning said number of days for cessation; and
calculating a current dose by subtracting a volume equal to said regression constant from a previous day's dose; and
replacing amount of vapor subtracted, by subtracting said regression constant from a previous day's dose, with vapor containing inert ingredients; and
continuing until the current dose is equal to zero, and vapor containing inert ingredients occupies the entire volume of vapor; wherein
the amount of vapor containing a substance remains constant during said control period and is gradually reduced to zero during said number of days for cessation.

7. The method for ceasing use of a substance of claim 6 further comprising:
delivering, for the duration of said control period, said initial dose by using the apparatus of claim 1 for the purpose of:
storing a volume of vaporizable liquid containing a substance; and
storing a volume of vaporizable liquid containing inert ingredients; and
wicking an amount of vaporizable liquid containing a substance; and
wicking an amount of vaporizable liquid containing inert ingredients; and
heating first wick until a first vapor forms for a set duration of a hit;
heating second wick until a second vapor forms to comprise the remainder of the hit; wherein
while the hit duration remains constant, the length of time in which the drug-containing vapor is delivered gradually reduces, and the length of time in which the vapor containing inert ingredients gradually increases, until the hit comprises 100 percent vapor containing inert ingredients.

8. The method for cessation of the use of a substance of claim 7 further comprising:
heating said first wick of the apparatus of claim 1 until a vapor forms; and
measuring the amount of heat applied to said first wick; and
calculating the amount of substance in said formed vapor from said first wick as a factor of heat applied and concentration of substance in said vaporizable liquid; and
heating said second wick of the apparatus of claim 1 until a vapor forms; and
measuring the amount of heat applied to said second wick; and
calculating the amount of inert ingredients in said formed vapor from said second wick as a factor of heat applied to said vaporizable liquid containing inert ingredients; and
reducing the calculated amount of substance in said vapor from said first wick and increasing the amount of inert ingredients in said vapor from said second wick by a set amount daily; wherein
the vapor is inhaled; and the vapor gradually contains less substance and more inert ingredients until the vapor contains only inert ingredients.

9. A method for ceasing use of a substance comprising:
a user interface for data entry of a user profile by a patient; and
said user profile comprising:
patient's history of substance use; and
patient's daily dose of a substance; and
patient's number of hits per day; and
intended date of cessation; and
information derived from data entry is converted to non-transitory computer-readable medium storing instructions; and
said instructions create a cessation plan by a linear equation; and
said instructions determine a control period; and
said instructions determine an initial dose; and
said instructions determine a regression constant; and
said instructions are uploaded to the apparatus of claim 1; wherein
said microprocessor carries out said instructions to provide measured initial dose during said control period and to subtract said control constant from each previous day's dose for the remainder of said cessation plan period and replace vapor containing said substance with vapor containing inert ingredients, continuing until all of said vapor in each of said hits per day is made up of inert ingredients.

10. The apparatus of claim 9 further comprising:
said instructions recording changes in atmospheric pressure as measured by said atmospheric pressure sensor; and
a substantial change in atmospheric pressure in said first body denotes the inhalation of vapor from said apparatus; and each event of inhalation of vapor is counted as one hit; and
said instructions determine the average number of hits per day during the control period; and
a significant increase in the number of hits in one day above said average number of hits initiates said instructions to contact an administrator of said cessation plan.

11. The apparatus of claim 9 further comprising:
said instructions recording changes in atmospheric pressure as measured by said atmospheric pressure sensor; and
a substantial change in atmospheric pressure in said first body denotes the inhalation of vapor from said apparatus; and each event of inhalation of vapor is counted as one hit; and
said instructions determine the average number of hits per day during the control period; and
a significant increase in the number of hits in one day above said average number of hits initiates said instructions to return to the most recent date wherein the number of hits per day were substantially equivalent to the average number of hits; and
a new end date is calculated by adding the number of days between the current date and the most recent date in which the number of hits per day were substantially equivalent to the average number of hits, to the current end date, to determine the new end date; and a new cessation plan is created using said linear equation; wherein
an adjustment is made in the cessation plan when the user deviates from the plan.

12. A method for ceasing use of a substance comprising:
a user interface for data entry of a user profile by a patient; and
said user profile comprising:
patient's history of substance use; and
patient's daily dose of a substance; and
patient's number of hits per day; and
intended date of cessation; and
information derived from data entry is converted to non-transitory, computer-readable medium storing instructions; and
said instructions create a cessation plan by an exponential equation; and
said instructions determine a control period; and
said instructions determine an initial dose; and
said instructions determine a regression constant; and
said instructions are uploaded to the apparatus of claim 1; wherein
said microprocessor carries out said instructions to provide measured initial dose during said control period and to calculate an exponential change in the dose for the remainder of said cessation plan period and replace vapor containing said substance with vapor containing inert ingredients; continuing until all of said volume of vapor in each of said hits per day is made up of inert ingredients.

13. The apparatus of claim 12 further comprising:
said instructions recording changes in atmospheric pressure as measured by said atmospheric pressure sensor; and
a substantial change in atmospheric pressure in said first body denotes the inhalation of vapor from said apparatus; and each event of inhalation of vapor is counted as one hit; and
said instructions determine the average number of hits per day during the control period; and
a significant increase in the number of hits in one day above said average number of hits initiates said instructions to decrease the absolute value of the regression constant; and
a new daily dose is calculated using the exponential formula; wherein
taking more than the average number of hits per day alters the instructions to accommodate user deviation in the cessation plan by reducing rapidity of the cessation plan.

14. The apparatus of claim 12 further comprising:
said instructions recording changes in atmospheric pressure as measured by said atmospheric pressure sensor; and
a substantial change in atmospheric pressure in said first body denotes the inhalation of vapor from said apparatus, and each event of inhalation of vapor is counted as one hit; and
said instructions determine the average number of hits per day during the control period; and
a significant decrease in the number of hits in one day below said average number of hits initiates said instructions to increase the absolute value of the regression constant; and
a new daily dose is calculated using the exponential formula; wherein taking less than the average number of hits per day alters the instructions to accommodate user deviation in the cessation plan by increasing the rapidity of the cessation plan.
